(19) United States
(12) Patent Application Publication
(10) Pub. No.: US 2013/0172246 A1
(43) Pub. Date: Jul. 4, 2013

(54) INSULIN GLARGINE VERSUS METFORMIN FOR THE FIRST-LINE TREATMENT OF EARLY TYPE-2 DIABETES

(71) Applicants: Wolfgang LANDGRAF, Frankfurt (DE); Markolf HANEFELD, Frankfurt (DE)

(72) Inventors: Wolfgang LANDGRAF, Frankfurt (DE); Markolf HANEFELD, Frankfurt (DE)

(21) Appl. No.: 13/692,640

(22) Filed: Dec. 3, 2012

Related U.S. Application Data
(60) Provisional application No. 61/682,060, filed on Aug. 10, 2012.

(30) Foreign Application Priority Data
Dec. 1, 2011 (EP) 11306600.5

(51) Int. Cl. A61K 38/28

(52) U.S. Cl. CPC A61K 38/28
USPC 514/5.9

(57) ABSTRACT

The invention relates to a method for treating type 2 diabetes patients, wherein insulin glargine is administered to the patient in an amount clinically tested to be safe and effective, wherein the type 2 diabetes is early type 2 diabetes, more particularly, wherein the insulin glargine is used as first-line drug.

The invention relates to a method for treating type 2 diabetes patients, wherein insulin glargine is administered to the patient in an amount clinically tested to be safe and effective, wherein the type 2 diabetes is early type 2 diabetes, more particularly, wherein the insulin glargine is used as first-line drug.

International guidelines recommend a lifestyle change plus metformin as the first option for the treatment of type 2 diabetes [1]. However, according to the data from the UK-PDS, metformin cannot stop the chronic progression of diabetes [2]. On the contrary, after a short time, there is a renewed increase in HbA1c and a continuous deterioration of β-cell function [3]. A substantial contribution to this is due to the insufficient effect of metformin on postprandial hyperglycemia [4]. Initial work by Monnier et al. [5] involving continuous blood glucose monitoring (CGMS) over 24 hours shows that the deterioration of diabetes firstly begins with a progressive rise in postprandial hyperglycemia in the morning after breakfast and only lastly includes the hours of the night when HbA1c has risen to >8.5%.

As can be seen from results from the UK-PDS and the DIG study [6], over 50% of the metformin-treated patients additionally received sulfonylureas or other combinations with oral antidiabetics after one to two years. However, for the combination of metformin plus long-acting sulfonylureas, increased cardiovascular mortality has to be expected [7]. Compared with this, insulin glargine can be used to achieve a stable HbA1c level of around 6% without serious adverse reactions such as severe hypoglycemias. This suggests that early insulinization not only allows long-term HbA1c control in a close to normal range, but also protects β-cell function. In agreement with this, in a pilot study with insulin glargine, we were able to demonstrate with a CGMS, improved control of postprandial hyperglycemia compared to metformin. There is now a multiplicity of pathophysiological and clinical studies, which have been brought together in the new “IDF Guideline on Postmeal Glucose Management” [9], which provide evidence for the close link between postprandial hyperglycemia, oxidative stress, subclinical inflammation and endothelial function [10]. Here, insulin is of particular importance. Both the diabetes intervention study [11] and the recently published results of a follow-up study of the UK-PDS [12] show that an early intensified therapy can lower total mortality. In contrast, the current megatrials (ACCORD, ADVANCE, VADT) were not able to demonstrate any benefit of strict glycemic control by means of late introduction of intensified therapy [13-15]. To this end, the benefit and the safety of an early insulin therapy compared to metformin must also be further evaluated. To date, there are no studies which measure this in terms of CGMS data, which alone allow an exact assessment of postprandial glucose excursion and glycemic variability. Little is also known about the β-cell protection possibly associated with earlier insulin therapy, and possible pleiotropic effects on inflammation, endothelial function and the vascular wall. The GLORY study has now evaluated these complex relationships for the first time and reviewed the start of therapy according to the EASD/ADA algorithm.

The trial is used to optimize therapy in the early phase of type 2 diabetes. Therapy up to now with metformin was not able to prevent the progression of diabetes. The control of postprandial glucose excursion using metformin is insufficient. To date, the long-term prevention of the progression of diabetes was possible only with insulin. The underlying mechanisms have been insufficiently explored to date. Our working hypothesis is as follows: near-normal setting of the glucose triad (fasting blood glucose, postprandial hyperglycemia and blood glucose fluctuations) achieves, in the long term, optimal glycemic control, which prevents the progression of diabetes. A priori, this also reduces the risk of hypoglycemia and prevents a greater increase in weight. In this connection, the protection of the beta-cells from glucotoxicity plays a central role. Furthermore, pleiotropic effects on inflammation and the endothelium are to be expected, which ultimately, in the long term, lead to a reduction in diabetes-related complications and in mortality, as has been evidenced by the DIS study [11] and the long-term data of the UK-PDS [12]. Currently, both the ADA/EASD and the DDG recommend a lifestyle change plus metformin for the initial therapy of type 2 diabetes. For metformin, there are decades of comprehensive data material relating to effectiveness and safety of Cochrane class 1a. However, this does not alter the fact that metformin, after a short-term improvement, leads to a continuous deterioration of the metabolic status, which has also been presently evidenced in the ADOPT study [16]. Metformin is also not tolerated in approximately 15% of cases [17] and has a range of contraindications. This is particularly the case for renal function disorders and heart failure (see product description). The demonstration of superior efficacy of insulin glargine therapy for the control of postprandial hyperglycemia and glycemic variability without increased risk of hypoglycemia is of huge clinical relevance. It opens the possibility of an alternative first-line therapy for patients who do not tolerate metformin or for whom there are metformin contraindications. The critical review of the ADA therapy algorithm is urgently necessary. Specifically the negative results of the ACCORD and VADT study in advanced diabetes with HbA1c values above 8% over many years and the long-term positive results of the DIS and UK-PDS study in newly diagnosed diabetics with intensified therapy have shown that the early intervention which stops the progression of diabetes at a low starting level is, in the long term, the only successful way at present of effectively reducing morbidity and mortality in type 2 diabetes.

Insulin glargine can be administered as basal insulin as a bedtime injection, and is thus not burdensome; this also applies to care personnel and elderly people in the family.

The patients are each assigned after randomization to insulin therapy or metformin. Since insulin is administered as an injection and the patient has to perform a dose adjustment on the basis of the current blood glucose level, blinding is not possible.

The trial was carried out with two established medicaments, which have been implemented in diabetes therapy for a long time and are used worldwide in millions of patients. For metformin, stomach complaints and also disturbances in appetite and, very rarely, allergies can occur in approximately 10% of patients. Since the use of metformin concerns the generally accepted indication, an improved metabolic control without significant risk is to be expected for the patient. Expected of insulin glargine are an additionally improved control of postprandial hyperglycemia, correction of endothelial dysfunction and improved protection of the β-cells. The patient thereby receives an additional benefit for his or her diabetes control with pleiotropic effects on a broad spectrum of cardiovascular risk factors. The dosing of insulin is carried out gradually according to the algorithm of the ORIGIN study, the largest insulin study worldwide with a now over three-year term. Principle risks of insulin therapy are hypoglycemia and increase in weight. However, in the case of the expected insulin doses of 0.3-0.4 U/kg of body weight, the risk of hypoglycemia is low. The same applies to increase in weight. A positive benefit/risk balance is thereby also to be expected for insulin. The investigation does not involve any risky procedures. For the collection of venous blood, the known rare local complications are to be expected. The same applies in principle to the subcutaneous application of the catheter of the CGMS system on the abdomen. Said catheter barely interferes with the patient. Local reactions and infections are very rare. The investigation does not involve any genetic analyses. All information is recorded and documented in pseudonymous form.

The goal of the trial was to evaluate the effectiveness and safety of a therapy with the basal insulin glargine compared to metformin as first-line drug in type 2 diabetes patients not diet-controlled in the HbA1c target range (6.5%).

The primary target variable determined was the Area Under the Curve (AUC) in mmol/L/time in subcutaneous abdominal fat after a test meal (TM) in a 2-hour course, which was determined by continuous blood glucose monitoring. The test meal was administered on the 2nd day of the CGM as breakfast.
AUC [mmol/L/time] 2 h pp

The secondary goals which we will investigate should provide information about whether an early insulin therapy can stop the progression of the deficit of β-cell function. Secondly, the effect on cardiovascular risk factors and endothelial function (or elasticity of the vascular wall) should be shown. In the case of HbA1c levels below 8%, the variability of HbA1c is dependent to >60% on postprandial glucose excursion. The pp hyperglycemia is a highly significant determinant of glucotoxicity for the β-cell, endothelial function and vascular risk. In connection with this, glycemic variability was calculated over 48 hours. This shows a close relationship with oxidative stress and risk of hypoglycemia. AUC and glucose variability, as sensitive parameters of the quality of diabetes control and of the progression/nonprogression of diabetes, can be expected to provide reliable conclusions with regard to the therapeutic benefit and the safety of the early insulin therapy.

Nowadays, CGM offers the possibility of recording latent hypoglycemias. Our study thereby makes an important contribution to evaluating possible risks owing to low blood glucose, especially at night. Also of interest is the anti-inflammatory action of insulin and the effect on renal function.

Below is a list of the various target parameters:
Protection of the β-cells by early insulin therapy
   C-Peptide (fasting and 2 h pp after TM)
   Proinsulin (fasting and 2 h pp after TM)
Glycemic variability:
   Glycemic load
   Standard deviation (SD),
   MAGE
   HbA1c
Anti-inflammatory action of insulin:
   Biomarkers of subclinical inflammation (fasting and 2 h pp)
      hsCRP,
      PAI-1,
      Leukocyte count
   Back-up samples for determining
      MMP9
      ADMA
      Adiponectin
Effect on endothelial function
   Endothelial function at ball of thumb using laser Doppler (O2C)
Effect on renal function
   Microalbuminuria (albumin/creatinine ratio)
   Serum creatinine
Risk of hypoglycemia
   Hypoglycemic intervals (time<3 mmol glucose) during CGM over 48 hours (day 2+3 after TM)
Free fatty acids

Within the study carried out, it has now been found that, surprisingly, early treatment of type 2 diabetes patients with GLA led to more effective control of the so-called “fasting glucose” level and of the overall glycemic situation with an improvement in beta-cell function compared to the standard treatment with MET. There was no rise in the number of hypoglycemic events. Therefore, the use of insulin glargine as first-line drug offers advantages over a corresponding administration of metformin.

The invention provides a method for treating type 2 diabetes patients, wherein insulin glargine is administered to the patient in an amount clinically tested to be safe and effective, wherein the type 2 diabetes is early type 2 diabetes.

The invention further provides a method as described above, wherein the insulin glargine is used as first-line drug.

The invention further provides a method as described above, wherein the diagnosis of the patient with type 2 diabetes is less than five years before the start of the treatment with insulin glargine. Preferably, the diagnosis may date back only four years, particularly preferably only three years and very particularly preferably only two years.

The invention further provides a method as described above, wherein the HbA1c level of the patient at the time of the diagnosis is between approximately 6.5 to approximately 8.5% inclusive. Preferably, the HbA1c level may also be between approximately 7.0 and approximately 8.0 inclusive.

The invention further provides a method as described above, wherein the dose of insulin glargine is determined according to a standardized titration algorithm for a target value of the fasting glucose level (FPG) of approximately 5.6 mmol/l or less.

Preferably, the target value of the fasting glucose level (FPG) of approximately 5.0 mmol/l or less may be determined.

The invention further provides a method for treating type 2 diabetes patients, wherein insulin glargine is administered as first-line drug to the patient in an amount clinically tested to be safe and effective, wherein the patient would have received an oral antidiabetic as first-line drug in a standard treatment.

The invention further provides a method as described above, wherein the diagnosis of the patient with type 2 diabetes is less than five years before the start of the treatment with insulin glargine. Preferably, the diagnosis may date back only four years, particularly preferably only three years and very particularly preferably only two years.

The invention further provides a method as described above, wherein the HbA1c level of the patient at the time of the diagnosis is between approximately 6.5 to approximately 8.5% inclusive. Preferably, the HbA1c level may also be between approximately 7.0 and approximately 8.0 inclusive.

The invention further provides a method as described above, wherein the dose of insulin glargine is determined according to a standardized titration algorithm for a target value of the fasting glucose level (FPG) of approximately 5.6 mmol/l or less. Preferably, the target value of the fasting glucose level (FPG) of approximately 5.0 mmol/l or less may be determined.

The invention further provides a method as described above, wherein the oral antidiabetic is selected from a group comprising metformin.

The invention further provides a method for treating type 2 diabetes patients, wherein insulin glargine is administered to the patient in an amount clinically tested to be safe and effective, wherein the patient had not yet received beforehand any insulin or insulin analog or oral antidiabetic.

The invention further provides a method as described above, wherein the diagnosis of the patient with type 2 diabetes is less than five years before the start of the treatment with insulin glargine. Preferably, the diagnosis may date back only four years, particularly preferably only three years and very particularly preferably only two years.

The invention further provides a method as described above, wherein the HbA1c level of the patient at the time of the diagnosis is between approximately 6.5 to approximately 8.5% inclusive. Preferably, the HbA1c level may also be between approximately 7.0 and approximately 8.0 inclusive.

The invention further provides a method as described above, wherein the dose of insulin glargine is determined according to a standardized titration algorithm for a target value of the fasting glucose level (FPG) of approximately 5.6 mmol/l or less. Preferably, the target value of the fasting glucose level (FPG) of approximately 5.0 mmol/l or less may be determined.

The invention further provides a method as described above, wherein the oral antidiabetic is selected from a group comprising metformin.

The invention further provides a method as described above, wherein the insulin or insulin analog is selected from a group comprising human insulin, insulin glargine, insulin detemir and insulin degludec.

The invention further provides a product comprising a packing material, insulin glargine and a label and/or a package leaflet in which it is indicated that patients who are being treated with insulin glargine can receive said treatment for early type 2 diabetes.

The invention further provides a product as described above, wherein the label and/or the package leaflet indicate that insulin glargine can be used as first-line drug.

### BRIEF DESCRIPTION OF THE DRAWINGS

FIG. 1 is a chart representing the treatment regime followed in Examiner 2.

The terms “type 2 diabetes” and “early type 2 diabetes” are used in the sense known in the field to all persons skilled in the art. The term “first-line drug” is likewise used in the sense known in the field to all persons skilled in the art.

The term “HbA1c level” refers to glycohemoglobin (GHb), which is hemoglobin bound to glucose. The HbA1c level is a measure of the mean blood glucose level of the last eight weeks (mean age of erythrocytes) and is therefore also referred to as long-term blood glucose or blood glucose memory.

The proportion of said HbA1c with respect to total hemoglobin is analyzed in individuals suffering from diabetes mellitus in order to monitor treatment outcome. The HbA1c is determined from whole blood, which usually comprises EDTA as anticoagulant. The level is measured nowadays using mainly laboratory machines, which fully automate the measurement process. The latest instruments use light-emitting diodes instead of a halogen lamp.

The term “standardized titration algorithm” refers to the algorithm mentioned in the introduction and known to a person skilled in the art, and can be found in the authorization documents of Lantus®.

The term “fasting glucose level (FPG)” is used in the sense known in the field to all persons skilled in the art.

The clinical experiments carried out are intended to more particularly elucidate hereinafter the inventive concept without having a restrictive effect. Examples 1-4 concern the study protocol of the underlying clinical study and are retained in the present form. Example 5 summarizes the methods used and example 6 reports in detail the results obtained. In example 7, a summary of the results can be found, and in example 8, the resulting conclusions.

### EXAMPLE 1

### Description of the Clinical Trial

Study design: The study described here is a multicenter, randomized, prospective, open, actively controlled, two-arm, parallel trial of Phase IV.
Patient number: For this clinical trial, a ratio of screened to randomized patients of 3:1 is accepted. A total of 100 patients, including an assumed drop-out rate of 20%, are included (randomized) in the trial, 50 thereof in the insulin glargine group and 50 thereof in the metformin group. It is not intended to replace drop-outs after randomization.

### Schedule:

Intended duration of the recruitment phase: 6 months
Duration of the therapy phase: 36 weeks

### Trial Population

### Inclusion Criteria

1. Written consent of the participating individual after provision of information
2. Patient with known type 2 diabetes (early stage: <5 years)
3. Male and female patients aged≧35≧75 years
4. HbA1c≧6.5≦8.0%, or ≦8.5% in the case of earlier administration of OAD

### Exclusion Criteria

1. Treatment with >1 oral antidiabetic (OAD) in the past
2. Treatment with 1 OAD within the last 6 weeks prior to randomization
3. Previous or current insulin treatment
4. Acute coronary syndrome<6 months
5. Severe liver disease (liver ASAT and/or ALAT levels>2.5 times the normal level) or liver failure, acute alcohol intoxication and or alcoholism;
6. Severe kidney disease or impairment of renal function (creatinine clearance<60 ml/min or creatinine>1.2 mg/dl)
7. Acute states which can lead to an impairment of renal function, for example: dehydration, severe infections, or shock;
8. Treatment with systemic, oral or injectable corticosteroids
9. Diseases and therapies which, at the discretion of the physician connected with the trial, cause the patient to appear unsuitable for participation in the study.
10. Intravascular administration of iodine-containing contrast agents
11. Diabetic ketoacidosis, diabetic precoma;
12. Acute or chronic diseases which can lead to tissue hypoxia, such as cardiac or respiratory failure, fresh myocardial infarction, shock;
13. Known history of hypersensitivity to one of the medicaments used or the ingredients or constituents thereof or to medicaments of similar chemical structure
14. Indications that the patient is unlikely to keep to the trial plan (e.g., unwillingness to cooperate)
15. Participation of the patient in another clinical trial within the last 4 weeks before inclusion (i.e., successful screening).
16. Alcohol and/or drug addiction—or other conditions which do not allow the individual affected to appreciate the nature and significance and also possible consequences of the clinical trial
17. Pregnant women or breast-feeding mothers
18. Women of child-bearing age, with the exception of women who meet the following criteria:
   Regular use of a contraceptive method with an error rate of <1% per year (e.g., implants, depot injections, oral contraceptives, intrauterine pessary-IUP).
   It should be taken into consideration that the combined oral contraception—in contrast to progesterone-only preparations—has a failure rate of <1%. Hormonal coils are safer than copper coils, with a Pearl Index of <1%.
   Postoperatively (6 weeks after double ovariectomy with or without hysterectomy)
      Patient recruitment: Firstly, patients shall come from the pool of already known patients of the centers and from the existing physician network of the centers. Secondly, patients shall have their attention drawn to the study via placed advertisements. Advertisements are to be submitted to the ethics commission for evaluation and may only be published after approval by the ethics commission.

### EXAMPLE 2

### Trial Preparation

Description of the trial preparation: Insulin glargine (trade name Lantus®) is a worldwide authorized insulin analog which is obtained from Escherichia coli by means of gene technology and has been on the market in Germany for over five years. The manufacturer is Sanofi-Aventis. It is available in pharmacies and from the manufacturer as a solution for injection with 100 units/ml in one prefilled pen. One pen contains 3 ml, corresponding to 300 units. Lantus® is a clear colorless solution which is stable for three years in a refrigerator. Prior to use of the prefilled pen (SoloStar®), the package leaflet must be studied carefully. The usage itself is not complicated. Insulin glargine is administered subcutaneously as a bedtime injection.

The most important possible adverse reactions of insulin glargine may be hypoglycemias and increase in weight. However, this applies especially to patients with higher starting levels of HbA1c (savings effect) and long-term diabetes. The tendency toward hypoglycemia can be minimized by flexible dose adjustment. In addition, owing to the CGMS, there is subtle information about the circadian changes in blood glucose under “real life” conditions. Insulin treatment may be associated with an undesired increase in body weight. Therefore, the patients receive an intensive consultation on nutrition at the start of the study. During the study, too, there is continuous advice on dose adjustment. The risk of an increase in weight is low. A range of medicaments for treating comorbidities, especially antihypertensives (ACE inhibitors, ARBs, beta blockers), can influence the blood glucose-lowering action or the sensation of shock.

Hypoglycemias are prevented as far as possible by the SMBG and flexible dose adjustment. The patients are encouraged not to change, if possible, all comedication during the study. The patients are advised that they ought to quickly inform the physician/nurse connected with the study in the event of any changes in medicament intake.

Comparative medication: Metformin is used as comparative medication. The tablets are made available to the patient in the center as a 500 mg, 850 mg and 1000 mg single dose. Metformin is the first-line drug recommended by the DDG and IDF when the diabetes cannot be kept in the target range solely by a lifestyle change.

Manufacture and labeling: This study uses two medicaments (metformin and insulin glargine) which are authorized and already represented on the market. There is no blinding, since the insulin glargine has to be injected subcutaneously. Both medicaments are labeled by the manufacturer as study medication.

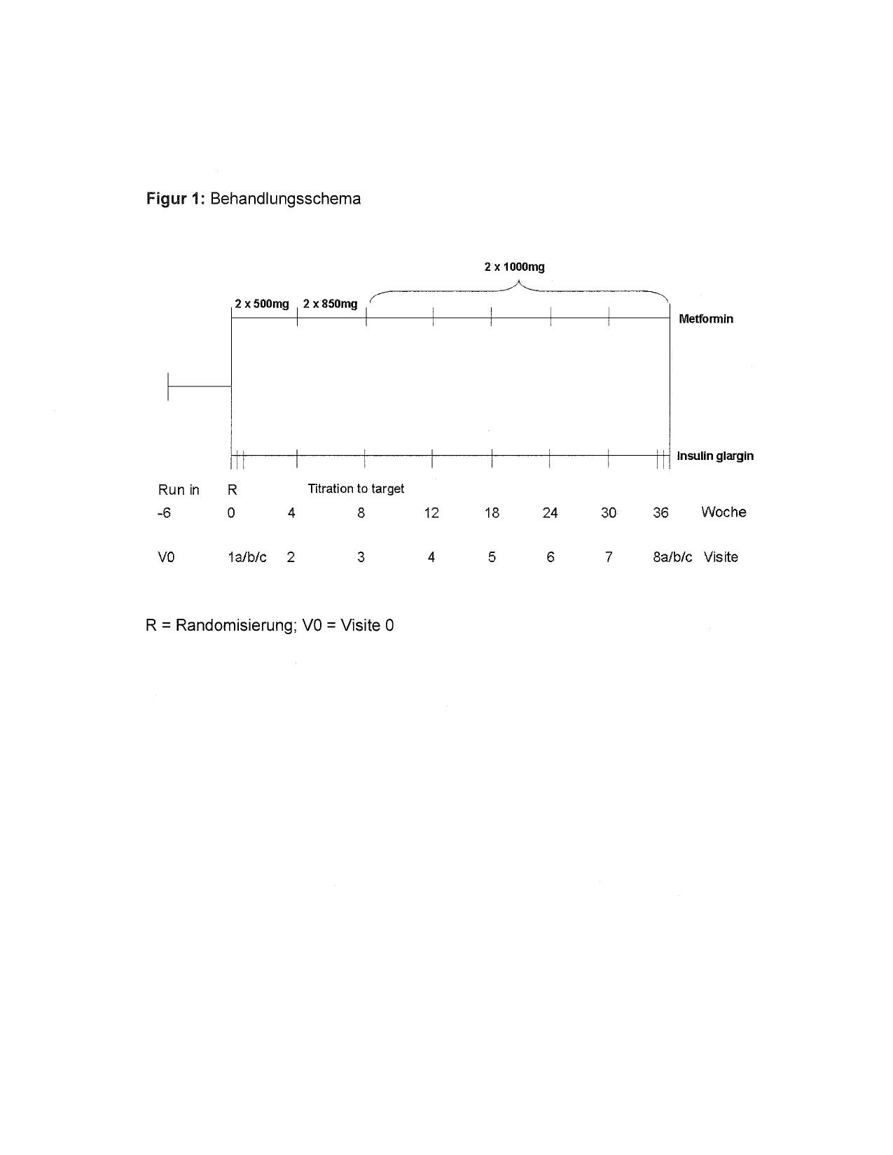

Randomization: On visit 0, a consecutive screening number from a number block (e.g., 001 to 300) is assigned to each patient to be studied. After checking all inclusion and exclusion criteria and successful completion of the run-in phase, the patient is randomized on visit 1 according to a predetermined scheme and thus randomly assigned to treatment group A—insulin glargine or B—metformin. Cf. FIG. 1.

### Instructions for Administration

Insulin glargine is administered subcutaneously using a pen (SoloStar®). The dose is titrated in order to achieve a fasting plasma glucose level of <5.6 mmol/l (100 mg/dl). Insulin glargine is injected 1× per day in the evening before bedtime (bedtime injection). The first insulin administration is done in the evening after visit 1c. The duration of the therapy is 36 weeks until visit 8c. The handling and storage of the pen is explained to the patients in a training course on visit 1c.

Metformin is administered as a film-coated tablet. The initial dose consists in the administration of 500 mg twice daily (morning and evening) during or after a meal. The first intake of the trial medication is done in the evening after visit 1c. The dose has to be adjusted according to the directions in 5.9. The duration of the therapy is 36 weeks until visit 8c.

### Instructions for Dose Adjustment

Insulin glargine: The insulin doses are adjusted on the basis of fasting plasma glucose levels and capillary blood results. The HbA1c level at randomization is used as a reference level for all HbA1c levels of the following visits; they are likewise used for monitoring the BG management of the diabetic.

The titration of the insulin dose is determined by the physician connected with the trial. The titration target is a glucose level of 5.6 mmol/l (100 mg/dl) fasting plasma glucose or 5.3 mmol/l (95 mg/dl) in the case of capillary blood measurement. The attainment of the target levels is supported by self-monitoring and documentation by the patients (diary) and also by regular follow-up with the physician connected with the trial.

The following steps should be carried out in the case of the patients in the insulin glargine group:
Provision of lifestyle consultation
Daily injection of the insulin once a day by the patient in order to attain the above-mentioned titration targets
(The patient is normally started with the evening administration of insulin, but this can be altered during the study on the basis of individual circumstances).
Determining the first insulin dose:
   The basis of the first insulin dose is the plasma glucose level which was determined in the fasting state on visit 1b.
   Table 1 below is used as a basis to determine the insulin dose with which the patients start:

TABLE 1

| Fasting plasma glucose | | Insulin glargine - |
|---|---|---|
| [mmol/l] | [mg/dl] | dose [units] |
| ≦6.7 | ≦120 | 6 |
| 6.8-8.3 | 121-150 | 8 |
| >8.3 | >151 | 10 |

The patient's fasting blood glucose will be determined daily by the patient until two successive levels 5.3 mmol/l (95 mg/dl) are measured. Thereafter, blood glucose measurements twice a week are sufficient.

The physician connected with the trial should check with each patient whether he or she is able and willing to carry out the instructions for adjusting the insulin dose by himself or herself. If the patients should be capable of doing this, they are given guidance on independent titration at home on the basis of the titration algorithm. The titration table is then handed over to the patients. The self-titration is carried out in parallel to the telephone visits. Generally, the titration of the dose should be carried out by the trial center within the first 4 weeks of the study. The basis is table 2 below:

TABLE 2
Insulin glargine titration algorithm:

| | Diary check of the last 3 FBG levels | | Change in evening |
|---|---|---|---|
| | If ≧2 levels | | dose at least |
| | mmol/l | mg/dl | 1x/week by |
| Increase | 4.1-5.3 | 74-96 | 0 units |
| in dose | >5.3 | >96 | 1 unit ↑ |
| | >6.0 | >108 | 2 units ↑ |
| | >6.9 | >124 | 3 units ↑ |
| Reduction | <4.1 | <74 | 1 unit ↓ |
| in dose | ≦3.2 | ≦58 | 2 units ↓ |

Outside the visits, the physician or the nurse connected with the study/the diabetes adviser contacts the patient by phone on a weekly basis during the first 8 weeks after randomization. If the target levels are attained after week 8, then the patient is contacted by phone again only in about one month's time (ideally between the visits in the study center). If the fasting blood glucose levels of the patient are still outside the target range after week 8, then weekly telephone contact is continued until attainment of the target range. The telephone visits are documented for each patient in a titration protocol provided therefor. Also, the patients have the option of phoning the study center in the event of any questions.

In the event of an occurrence of hypoglycemia requiring help from a third party:
The patient should be given guidance on reducing the dose by at least 2 units.
The patient should contact the physician or the nurse connected with the study as soon as possible.
The patient should restart the titration after consultation with the study personnel.

To ensure compliance and therapy safety, each patient in the insulin glargine group receives a diary in which all the self-monitored blood glucose levels and also the insulin dose and possible adverse reactions are entered.

Metformin: The patients receive a lifestyle consultation at the start of the study and an initial dose of 500 mg of metformin twice daily as trial medication in the first four weeks. Each tablet is taken during breakfast and during supper, respectively, or immediately thereafter to prevent gastrointestinal complaints.

After 4 weeks (visit 2), the dose is increased to 2 times 850 mg if the patient has tolerated the initial dose. If the patient reports intolerable, persisting adverse reactions in connection with the trial medication, the administration of metformin is to be reduced to once daily on visit 2 and to be possibly increased again on the next visit.

After 8 weeks (visit 3), the dose is to be increased to 1000 mg twice daily if tolerable. In case of complications, the dose is reduced to the next lower dose. Metformin can lead to lactic acidosis in very rare cases if there are impairments of renal function. Possible conditions which increase the risk of lactic acidosis were incorporated in the protocol as exclusion criteria. In addition, the creatinine and liver levels over the course of the study are monitored.

Emergency measures: At the start of the intake of the trial medication, the patient receives a briefing on metformin intake or a training course on insulin therapy. The training course for the insulin glargine group shall contain advice on avoiding hypoglycemias and first aid measures in the event of low blood glucose. In line with the DDG (enclosure 8), the emergency measures include guidelines on treating diabetes mellitus:
In the event of slight hypoglycemias:
Oral administration of rapidly absorbable carbohydrates (1-2 carbohydrates in the form of sugar-containing beverages or glucose, for example 4 Dextrogen tablets)
In the event of severe hypoglycemias requiring outside help: Immediately inform the emergency physician, who initiates appropriate emergency treatment.

Concomitant medication: During the study, the patient should not take any additional medicaments which modify glucose metabolism, for example oral or parenteral corticosteroids, hydrochlorothiazide at a dose of 25 mg/day or beta blockers at more than one equivalent dose based on 40 mg of propanolol/day. If the administration of said medicaments should become necessary, for example in an emergency, the physician connected with the trial must be informed.

Compliance: The patient receives, on each visit, study medication which lasts until the next visit. Compliance with the study by the patients is checked by counting the study medication handed back on the corresponding visits. In the case of metformin, the blisters are counted, and in the case of insulin glargine, the vials are counted and checked to determine whether they have been used. Compliance is adhered to when at least 80% and not more than 120% of the medication to be taken has been taken by the patient.

If compliance is above or below this value, protocol violation has occurred and the patient must be excluded from the remainder of the study.

Moreover, the patient keeps a patient's diary, in which the medication taken shall also be recorded.

In the weekly telephone visit, the physician connected with the trial will discuss the intake of the medication.

### EXAMPLE 3

### Trial Procedure

The duration of the study, including the 6-week run-in phase, is 42 weeks. The patients who, following detailed information, have declared in writing their consent to participation in the study are subjected to a thorough clinical examination at the start of the study (visit 0). The result of the examination is used to assess the inclusion and exclusion criteria. Upon meeting said criteria, the patients receive a patient number, which is determined randomly, in visit 1. The study medication is administered in line with this number. There are no genetic analyses. The patients are informed that, for the duration of the study, they may not simultaneously participate in another study.

Additionally, an “autonomic nervous system” extension study, which is described in detail in appendix A with its own protocol, is carried out at the Dresden center.

### Visit 0 (Screening, Week-6)

Each individual must be comprehensively informed about the trial prior to admission to the clinical trial. The information is provided in person by the responsible physician and in writing by the patient information document. Only after clarification of all questions from the participating individual is he or she requested to sign two copies of the consent form and to date them personally. Subsequently, one copy of the patient information document/consent form is handed over to the participating individual; the second copy is kept in the investigator site file. Subsequently, the following data are to be collected:
Informing the patient, consent form
Checking the inclusion/exclusion criteria
Collecting demographic data (for example, ethnicity, sex, age, body weight, waist/hip measurement, height)
Medical history, recording concomitant medication and concomitant conditions
Vital signs (pulse, blood pressure),
Physical examination,
ECG
Clinical chemistry:
   Efficacy: HbA1c, fasting plasma glucose,
   Safety creatinine and albumin in urine, serum creatinine, ALAT, ASAT.
Pregnancy test, if applicable
Handing out SMBG devices and patient's diaries after briefing and training the patients in methods for self-monitoring of blood glucose (SMBG) and diary documentation

For patients with no previous medicinal diabetes therapy, the run-in phase can be shortened to 1 week (±5 days).

### Visit 1

### Visit 1a (Randomization, Week 0, 1st Day)

Checking the inclusion/exclusion criteria
Physical examination
Measuring body weight, waist/hip measurement, blood pressure, pulse
Recording the change in concomitant medication and concomitant conditions
Recording AEs and SAEs
Applying the CGMS for up to 72 h, briefing and training the patient
Randomization:
   Group A: Titrating insulin glargine to target level of fasting plasma glucose (5.6 mmol/l (100 mg/dl))
   Briefing and training the patients in insulin use (Depending on the organization, training measures can be implemented during the overall visit 1 a-c.)
   Group B: 500 mg of metformin tid
Pregnancy test, if applicable.
Laser Doppler measurement; only at center 01 and 02

### Visit 1b (Randomization, Week 0, 2nd Day)

Test meal
Clinical chemistry:
   Efficacy:
   HbA1c, insulin*, C-peptide*, proinsulin (intact)*, fasting plasma glucose*, triglycerides, total cholesterol, HDL cholesterol, LDL cholesterol, free fatty acids, hsCRP, PAI1 and leukocyte count*.
   (*)=Repetition 2 h pp,
   Safety
   Creatinine and albumin in urine, serum creatinine, ALAT, ASAT.
Recording AEs and SAEs

### Visit 1c (Randomization, Week 0, 4th Day)

Removal of CGMS, evaluating the diaries
Recording AEs and SAEs
Handing out trial medication according to randomization after briefing in medicament intake
Injection training
Advice for the patient: first evening injection of the determined glargine dose according to fasting plasma glucose of visit 1b.
Arranging the at first weekly telephone visits (further procedure of the telephone contacts depending on the attainment of the target range).

### Visit 2 (Titration Visit, Week 4, ±5 Days)

Measuring body weight, blood pressure, pulse
Recording the change in concomitant medication,
Recording AEs and SAEs
Clinical chemistry:
   Efficacy: HbA1c, fasting plasma glucose,
   Safety creatinine and albumin in urine, serum creatinine, ALAT, ASAT.
Trial medication:
   Group A: Titrating insulin glargine to target level of fasting plasma glucose (5.6 mmol/l (100 mg/dl))
   Group B: Increasing the dose to 850 mg of metformin tid if no contraindications
Compliance (registering the medication used up so far, handing out new trial medication)

### Visit 3 (Titration Visit, Week 8, ±5 Days)

Measuring body weight, blood pressure, pulse
Recording change in concomitant medication
Recording AEs and SAEs
Clinical chemistry:
   Efficacy: HbA1c, fasting plasma glucose,
   Safety creatinine and albumin in urine, serum creatinine, ALAT, ASAT.
Trial medication:
   Group A: Titrating insulin glargine to target level of fasting plasma glucose (5.6 mmol/l (100 mg/dl))
   Group B: Increasing the dose to 1000 mg of metformin tid if no contraindications
Drug accountability/compliance (registering the medication used up so far, handing out new trial medication)

### Visit 4 (Safety Visit, Week 12, ±5 Days)

Measuring body weight, blood pressure, pulse
Recording the change in concomitant medication
Recording AEs and SAEs
Clinical chemistry:
   Efficacy: HbA1c, fasting plasma glucose,
   Safety creatinine and albumin in urine, serum creatinine, ALAT, ASAT.
Drug accountability/compliance (registering the medication used up so far, handing out new trial medication)

### Visit 5 (Safety Visit, Week 18, ±5 Days)

Measuring body weight, blood pressure, pulse
Recording the change in concomitant medication
Recording AEs and SAEs
Clinical chemistry:
   Efficacy: HbA1c, fasting plasma glucose,
   Safety creatinine and albumin in urine, serum creatinine, ALAT, ASAT.
Drug accountability/compliance

### Visit 6 (Safety Visit, Week 24, ±5 Days)

Measuring body weight, blood pressure, pulse
Recording the change in concomitant medication and concomitant conditions
Recording AEs and SAEs
Clinical chemistry:
   Efficacy: HbA1c, fasting plasma glucose,
   Safety creatinine and albumin in urine, serum creatinine, ALAT, ASAT.
Drug accountability/compliance

Visit 7 (safety visit, telephone, week 30, ±5 days)
Interview by phone
Recording the change in concomitant medication and concomitant conditions
Recording AEs and SAEs
Requesting the blood glucose level (SMBG level)

### Visit 8

### Visit 8a (Concluding Visit, Week 36, ±5 Days, 1st Day)

Recording the change in concomitant medication
Recording AEs and SAEs
Physical examination
Measuring body weight, waist/hip measurement, blood pressure, pulse
Applying the CGMS for up to 72 h, briefing and training the patient
Drug accountability/compliance (registering the used-up medication)
Laser Doppler measurement, only at center 01 and 02

### Visit 8b (Concluding Visit, Week 36, 2nd Day)

Test meal
Clinical chemistry:
   Efficacy:
   HbA1c, insulin*, C-peptide*, proinsulin (intact)*, fasting plasma glucose*, triglycerides, total cholesterol, HDL cholesterol, LDL cholesterol, free fatty acids, hsCRP, PAI1 and leukocyte count*
   (*)=Repetition 2 h pp
   Safety
   Creatinine and albumin in urine, serum creatinine, ALAT, ASAT.
Pregnancy test
Recording AEs and SAEs

### Visit 8c (Concluding Visit, Week 36, 4th Day)

Removal of CGMS, evaluating the diaries
Recording AEs and SAEs
Return of unused trial medication, drug accountability/compliance determination
End of the treatment
Report for the family physician responsible for further treatment of the patient

In addition, weekly telephone visits are carried out (procedure of telephone contacts as described in 5.9.1). Here, the patients are questioned concerning their well-being and particular events, especially with respect to the medication. In the case of the patients of the insulin glargine group, the self-monitored blood glucose levels and the injected units are additionally discussed and, if necessary, advice is given with respect to the dose (units). Follow-up examinations

CGMS: The system used for continuous glucose monitoring is the CGMS from Medtronic, CGMS® System Gold™ monitor/MiniMed glucose subcutaneous sensor. The system is authorized for continuous glucose monitoring over a period of 72 h. The measurement sensor is inserted into subcutaneous fat tissue with the aid of an applicator. The entire sensor is stuck on the skin using a plaster. This is followed by the calibration of the device for approximately 5 hours, during which the subcutaneous levels are synchronized with the capillary blood glucose levels. Over the course of 72 h, further calibrations are required, as recommended by the manufacturer. The measured data of the sensor are transmitted by means of radio frequency to the CGMS® System Gold™ monitor, which is worn on the belt for example. After completion of the measurement period, the sensor is removed and the data of the monitor are evaluated on the computer using the software. For the evaluation, only the measured data of the first 48 hours are used, since the accuracy of measurement decreases over the course of the third day.

SMBG: Glucose is monitored from capillary blood (self-monitoring of blood glucose) using the FreeStyle Lite® from Abott Diabetes Care, Wiesbaden. The patients are taught the blood glucose monitoring methods (CGMS and SMBG) and educated in dealing with and documenting the results (e.g., hypoglycemia/hyperglycemia and patient's diary).

Test meal: On the morning of the 2nd day of the CGM, there is a standardized test meal in the trial center. This test meal (TM) consists of 95 g of whole-wheat bread, 20 g of margarine, 25 g of fruit preserves, 25 g of cheese, 200 ml of orange juice, 200 ml of milk mix with banana, strawberry or cocoa with a total of 560 Kcal. Blood is taken from the patient prior to the TM (after night-time fasting period of at least 8 hours). Subsequently, the TM should be eaten within 15 min. From the 1st bite, the time is measured, and after 2 h have elapsed, a 2nd blood collection is performed. During the TM and during the two hours thereafter, the patient should move as little as possible so as not to falsify the monitoring.

Fasting blood is collected between 7 and 9 o'clock in the morning when the patient turns up at the center.

Laser Doppler measurement (O2C): The method of combined laser Doppler fluximetry and white light spectrophotometry (O2C, LEA Medizintechnik, Giessen) Germany is used for the simultaneous measurement of microvascular blood flow and oxygen saturation in the skin. It is a noninvasive measurement in the region of the back of the hand. As depicted in the following FIGURE, a flat probe (LF2, LEA-Medizintechnik, Giessen) is fixed for this purpose onto the back of the hand in the region of the hypothenar eminence. Using a conventional blood pressure cuff, a suprasystolic stasis is generated for 4 minutes on the upper arm of the patient and this causes ischemia in the region of the tissue under the measurement probe. After opening of the suprasystolic stasis, the postischemic microvascular blood flow (LDF) and the oxygenation of the hemoglobin (sO2) and of a tissue depth of approximately 2 mm and of a tissue depth of approximately 8 mm is measured noninvasively. The measurement is carried out on one side of the body, and this side of the body is then to be retained for the patient over the course of the study. All devices used for this purpose are CE-certified and are used in routine diagnostics.

Laboratory analyses: The blood samples for the clinical laboratory tests are analyzed in the central laboratory and the local laboratories according to standardized conditions. The central laboratory and the local laboratories provide on request corresponding documentation (e.g., normal value tables, round-robin test certificates in accordance with RiLiBÄK (Guidelines of the German Medical Association)) for the trial master file. The central laboratory is responsible for the collection, transport and analysis of the blood samples and instructs the trial center on sample management.

### Parameters and Methods:

HbA1c chromatographically by HLPC (device: TOSOH A1c)
Plasma glucose by enzymatic UV test HK (device: Selectra)
Creatinine in urine by PAP test (enzyme-photometrically) (device: Selectra)
Albumin in urine (spontaneous) by immunoturbidimetric method (device: Selectra)
Microalbuminuria (calculated: albumin/creatinine ratio)
Serum creatinine enzyme-photometrically by means of creatinine PAP test (device: Selectra)
ALAT, ASAT by optimized UV method according to IFCC (device: Selectra)
Insulin by ELISA (solid-phase enzyme immunoassay; device: Anthos)
C-peptide by ELISA (solid-phase enzyme immunoassay; device: Anthos)
Proinsulin (intact) (solid-phase enzyme immunoassay; device: Anthos)
Triglycerides by GPO-PAP method (device: Selectra)
HDL cholesterol by enzymatic test (device: Selectra)
LDL cholesterol by enzymatic test (device: Selectra)
Free fatty acids by photometric method (NEFA) (device: Selektra)
hsCRP by immunoturbidimetric method (device: Selectra)
PAI-1 (plasminogen activator inhibitor active antigen) bioimmunoassay
Leukocyte count by electronic counting
Pregnancy test (urine dipstick)

Back-up samples for determining MMP9, ADMA, and adiponectin From the obtained back-up samples, MMP9, ADMA and adiponectin are to be additionally determined, if necessary, at a later time. These samples are kept for up to five years, and are then destroyed. The collection, preparation and storage directions of the laboratory are observed to ensure standardized conditions.

Applicable directions are to be supplied to the trial master file for the purposes of documentation.

Further treatment of the patients after completion of the clinical trial: during visit 8 (concluding visit), further therapy after the end of the study is discussed with the study participant.

When the last laboratory parameters collected during visit 8 are available, the study participant is sent a closing letter which contains all collected findings and any further therapeutic recommendations and which can be made available to his or her family physician.

### EXAMPLE 4

### Statistics

Sample size planning: It is known from the ORIGIN-CGMS substudy that the difference in the AUC TM for BG between the group with insulin glargine and the group with standard treatment (predominantly metformin therapy) is 15%, i.e., 127 mmol*120 min with a standard deviation of 179 mmol*120 min. The null hypothesis is:

H₀: μ_{glargine}=μ_{metformin,}

i.e., the mean reduction (end to base) of the AUC TM for BG under the therapy with insulin glargine is the same as the mean reduction under metformin.

The alternative hypothesis can be formulated by

H₁: μ_{glargine}<μ_{metformin,}

i.e., the mean values of the AUC TM are lowered by the insulin glargine therapy.

The type I error is determined by α=0.05, based on a two-tailed test for equality of the treatment types, and a type II error (power) is determined by 90%. Assuming that the sex distribution of the patients included in the study is the same, but possible sex-specific differences in the efficacy of the trial medication have to be considered, and with the estimation of a 20% drop-out rate, this gives rise to a sample size of 50 patients per treatment arm.

### Statistical Methods

### Primary Target Variable

The primary target variable is the AUC (BG) TM of the glucose concentration in subcutaneous abdominal fat after a standardized test meal over a period of 2 hours. The AUC is calculated accordina to the trapezoidal rule.

$$AUC ( BG ) TM := {\sum }_{i = 1}^{n} \frac{1}{2} ( {t}_{i} - {t}_{i - 1} ) ( {f}^{{BG}_{i}} + {f}^{{BG}_{i - 1}} )$$

### Secondary Target Variables

Mean HbA1c,
Glycemic variability:
   Standard deviation (SD) of the mean glucose concentration over 24 h (day 2),
   MAGE—mean amplitude of glycemic excursion calculated as the mean from the glucose increases>1 SD of the mean glucose concentration over the course of 24 h
Glycemic load calculated by (FG+Peak+2hppG)·⅓
Mean of C-peptide and intact proinsulin (fasting and 2 h pp)
Mean free fatty acids
Biomarkers of subclinical inflammation (hsCRP, PAI-1, leukocyte count fasting and 2 h postprandial)
Microalbuminuria (albumin/creatinine ratio)
Endothelial function at ball of thumb using laser Doppler (O2C)
Hypoglycemic intervals (time<3 mmol glucose) during CGM over 48 hours (day 2+3 after TM)

For the statistical evaluation, the data from the case report forms (CRF) are entered into a specific database. In the case of the first two CRFs, a complete data comparison is carried out after the date input. For the following CRFs, the checks are realized on a random basis. Plausibility checks are carried out for all parameters entered. All available data are included in the analysis of the results. Missing data are not replaced and considered to be missing values in the analysis. The LOCF approach is used where the use thereof is meaningful. A detailed description is effected in the SAP.

The program available for the statistical evaluation is SPSS version 17.0.

The analyses are carried out according to the per protocol scheme, i.e., all patients with complete data sets are included in the evaluation, severe protocol violations are excluded and according to the FAS.

### Definition of FAS and PPS:

[The full analysis—data (nFAS) set comprises all patients, who
were enrolled,
were randomized,
received at least one application of study medication,
post-randomization data of the primary target variable is available,

It is the set of subjects with treatment effects measured, according to the intention-to-treat principle. The corresponding analyses contain safety parameter and demographic data summaries.

The per protocol set (nPPS) consists of all subjects of the full analysis set without any major protocol violations. It is the set of subjects that participated in the trial as intended, according to the protocol.

The corresponding analyses contain supplementary considerations of the efficacy data.]

The demographic and medical history data collected as baseline and the laboratory parameters are compiled. The evaluation of the primary parameter is compared per arm (null hypothesis test) in the form of the paired t-test (values on visit 1 vs. visit 8).

The analysis includes all randomized patients from which evaluable CGMS values were collected on visit 1 vs. visit 8. The analysis of the influence of the study therapy on the primary target variable is carried out by covariance analysis (ANCOVA) taking account of the starting values for the AUC TM.

### EXAMPLE 5

### Summary of the Methods

In the study in the previous examples, 75 patients (45 men, 30 women, mean age: 60.7±9.2 years) were examined, whose first diagnosis of type 2 diabetes dated back to less than 5 years and whose HbA1c level was between 6.5 and 8.5%. The patients were treated with either metformin (MET, 1000 mg b.i.d. (n=3)) or insulin glargine (GLA) q.d. just before bedtime (n=3). The insulin dose was adjusted for a target plasma glucose concentration (FPG) of ≦5.6 mmol/L according to a standardized titration algorithm. At the start and end of the study, the HbA1c level and the plasma glucose, proinsulin and C-peptide concentrations were determined. Furthermore, continuous glucose monitoring (CGM) was carried out for over 72 hours (Medtronic, Germany) in order to determine the interstitial glucose level (IG) and the area under the curve (AUC). All the patients received a standardized test meal on the second day of the continuous glucose monitoring for comparison of the interindividual postprandial (pp) glucose excursions.

### EXAMPLE 6

### Results

### Evaluation of Effectiveness—Analyzed Data Sets

There were three defined data sets: full analysis set or intention to treat (FAS or ITT), per protocol set (PPS) and the safety set.

Included in the analysis were 96 patients who were randomized, received at least one application of a test medicament and for whom postrandomization of the primary target variable was available.

The per protocol (“PP”) set contained all study patients of the entire analysis without larger protocol variations, it is the set of the study patients who had participated in the study per protocol as originally planned. Patients were excluded from the PP set when at least one of the following conditions was met:
Inclusion and exclusion criteria were violated
Express wish of the patient to end or change the therapy
Application of excluded treatments

At the end of the study, there were 75 patients with complete AUC data and without violations of the protocol. For all the statistical analyses, the PPS was used.

The third data set for the analysis is the one with regard to the safety population. This population includes all study patients who had received the IMP or another treatment according to the study. This data set was used to validate the failure rates of the treatment and control groups.

The study results are shown in the following tables.

TABLE 3
Background information relating to all randomized patients
(ITT) in comparison with the metformin and insulin groups

| | All patients | Metformin | Insulin | Sign. |
|---|---|---|---|---|
| | (n = 96) | (n = 49) | (n = 47) | M vs. I |
| Age (years) | 61.4 (9.0) | 62.1 (9.2) | 60.6 (8.9) | 0.411 |
| Sex | | | | |
| Male | 57 (59%) | 26 (53%) | 31 (66%) | 0.198 |
| Female | 39 (41%) | 23 (47%) | 16 (34%) | |
| Diabetes duration | 2.8 (1.6) | 2.8 (1.7) | 2.8 (1.6) | 0.945 |
| (years) | | | | |
| HbA_{1C} (%) | 7.0 (0.6) | 6.9 (0.4) | 7.2 (0.7) | 0.021² |
| BMI (kg/m²) | | | | |
| Male | 29.2 (4.4) | 29.5 (4.6) | 28.8 (4.2) | 0.557 |
| Female | 31.5 (5.2) | 31.0 (5.5) | 32.4 (4.6) | 0.406 |
| Hip measurement | | | | |
| (cm) | | | | |
| Male | 106.5 (9.2) | 107.1 (9.8) | 106.0 (8.8) | 0.655 |
| Female | 112.7 (11.8) | 111.5 (11.7) | 114.6 (12.0) | 0.431 |
| Abdominal circ. | | | | |
| (cm) | | | | |
| Male | 105.2 (11.4) | 105.3 (10.5) | 105.1 (12.4) | 0.964 |
| Female | 102.9 (13.9) | 100.7 (15.0) | 106.3 (11.5) | 0.222 |
| Blood pressure | | | | |
| Systolic (mmHG) | 140.8 (15.6) | 141.6 (15.1) | 139.9 (16.1) | 0.598 |
| Diastolic (mmHG) | 83.4 (10.4) | 81.9 (10.9) | 85.0 (9.7) | 0.151 |
| Heart rate (1/min) | 70.8 (11.0) | 69.2 (11.0) | 72.5 (10.7) | 0.138 |
| Albumin/creatinine | 3.2 (11.8) | 2.3 (2.8) | 4.3 (16.7) | 0.842² |
| ratio (mg/mmol) | | | | |
| Total chol. | 5.2 (1.0) | 5.1 (1.0) | 5.2 (1.1) | 0.718 |
| (mmol/l) | | | | |
| Triglycerides | 2.1 (1.4) | 1.9 (1.3) | 2.4 (1.5) | 0.058² |
| (mmol/l) | | | | |
| HDL chol. | 1.2 (0.4) | 1.3 (0.4) | 1.1 (0.3) | 0.048 |
| (mmol/l) | | | | |
| LDL chol. (mmol/l) | 3.1 (0.8) | 3.1 (0.8) | 3.1 (0.8) | 0.748 |
| hsCRP (mg/l) | 3.0 (4.1) | 3.0 (4.8) | 3.0 (3.2) | 0.649² |
| Plasma glucose | | | | |
| (mmol/l) | | | | |
| Fasting | 8.9 (1.9) | 8.6 (1.5) | 9.3 (2.2) | 0.103 |
| 2 h after test meal | 10.7 (3.8) | 10.3 (2.8) | 11.2 (4.6) | 0.302 |
| Leukocytes (GPtl/l) | 6.3 (1.6) | 6.4 (1.5) | 6.3 (1.8) | 0.704 |
| Insulin (mU/l) | | | | |
| Fasting | 12.0 (7.7) | 12.1 (8.1) | 11.9 (7.4) | 0.920 |
| 2 h after test meal | 60.3 (40.7) | 63.6 (42.1) | 56.7 (39.5) | 0.420 |
| Proinsulin (pmol/l) | | | | |
| Fasting | 10.8 (10.1) | 8.9 (6.0) | 12.8 (12.9) | 0.029² |
| 2 h after test meal | 33.0 (25.1) | 28.3 (16.6) | 37.9 (31.2) | 0.070 |
| HOMA | 48.2 (34.0) | 48.9 (32.8) | 47.5 (35.7) | 0.854 |
| C-peptide (nmol/l) | 2.8 (1.3) | 2.9 (1.4) | 2.7 (1.3) | 0.602 |

²log-transformed for t-test

TABLE 4
Background information relating to all “per protocol” (PP)
patients in comparison with the metformin and insulin groups

| | All patients | Metformin | Insulin | Sign. |
|---|---|---|---|---|
| | (n = 75) | (n = 36) | (n = 39) | M vs. I |
| Age (years) | 61.2 (9.2) | 62.5 (9.1) | 60.0 (9.3) | 0.244 |
| Sex | | | | |
| Male | 45 (60%) | 19 (53%) | 26 (66%) | 0.220 |
| Female | 30 (40%) | 17 (47%) | 13 (34%) | |
| Diabetes duration | 2.9 (1.6) | 2.8 (1.6) | 3.0 (1.6) | 0.619 |
| (years) | | | | |
| HbA_{1C} (%) | 7.1 (0.6) | 6.9 (0.4) | 7.2 (0.7) | 0.054² |
| BMI (kg/m²) | | | | |
| Male | 29.0 (4.6) | 29.9 (5.1) | 28.3 (4.1) | 0.256 |
| Female | 32.2 (5.5) | 30.8 (6.3) | 31.6 (4.5) | 0.685 |
| Hip measurement | | | | |
| (cm) | | | | |
| Male | 106.5 (9.9) | 108.3 (10.9) | 105.2 (9.1) | 0.317 |
| Female | 111.7 (12.5) | 110.9 (13.3) | 112.8 (11.7) | 0.698 |
| Abdominal circ. | | | | |
| (cm) | | | | |
| Male | 104.3 (12.0) | 105.1 (12.0) | 103.7 (12.2) | 0.720 |
| Female | 101.4 (14.1) | 99.7 (17.1) | 103.6 (9.0) | 0.421 |
| Blood pressure | | | | |
| Systolic (mmHG) | 141.3 (15.3) | 141.7 (14.9) | 141.0 (15.7) | 0.857 |
| Diastolic (mmHG) | 83.6 (10.3) | 81.7 (10.5) | 85.3 (9.8) | 0.128 |
| Heart rate (1/min) | 71.2 (10.6) | 69.5 (10.3) | 72.7 (10.8) | 0.203 |
| Albumin/creatinine | 3.2 (12.9) | 2.1 (2.9) | 4.2 (17.7) | 0.785² |
| ratio (mg/mmol) | | | | |
| Total chol. | 5.3 (1.1) | 5.3 (1.0) | 5.3 (1.1) | 0.834 |
| (mmol/l) | | | | |
| Triglycerides | 2.2 (1.5) | 2.1 (1.4) | 2.4 (1.6) | 0.440² |
| (mmol/l) | | | | |
| HDL chol. (mmol/l) | 1.2 (0.4) | 1.3 (0.4) | 1.2 (0.3) | 0.187 |
| LDL chol. (mmol/l) | 3.2 (0.8) | 3.2 (0.8) | 3.2 (0.8) | 0.991 |
| hsCRP (mg/l) | 3.2 (4.4) | 3.4 (5.5) | 3.1 (3.3) | 0.904² |
| Plasma glucose | | | | |
| (mmol/l) | | | | |
| Fasting | 9.0 (1.9) | 8.7 (1.7) | 9.2 (2.1) | 0.297 |
| 2 h after test meal | 10.7 (3.8) | 10.3 (2.8) | 11.1 (4.5) | 0.406 |
| Leukocytes (GPtl/l) | 6.2 (1.6) | 6.3 (1.6) | 6.1 (1.6) | 0.706 |
| Insulin (mU/l) | | | | |
| Fasting | 12.2 (8.2) | 12.7 (9.0) | 11.9 (7.6) | 0.687 |
| 2 h after test meal | 59.3 (42.2) | 63.0 (43.2) | 56.1 (41.6) | 0.482 |
| Proinsulin (pmol/l) | | | | |
| Fasting | 11.1 (10.9) | 8.8 (6.3) | 13.0 (13.6) | 0.035² |
| 2 h after test meal | 32.9 (26.2) | 27.4 (16.0) | 37.8 (32.2) | 0.086 |
| HOMA | 48.8 (35.4) | 49.5 (34.5) | 48.3 (36.7) | 0.893 |
| C-peptide (nmol/l) | 2.9 (1.4) | 3.0 (1.5) | 2.8 (1.3) | 0.453 |

²log-transformed for t-test

TABLE 5
Data for all randomized patients (ITT) divided according to study site

| | Site 1 | Site 2 | Site 3 | Site 4 | Sign. |
|---|---|---|---|---|---|
| | (n = 68) | (n = 8) | (n = 6) | (n = 14) | M vs. I |
| Age (years) | 62.5 (8.3) | 59.5 (12.7) | 60.8 (8.7) | 57.5 (10.2) | 0.271 |
| Sex | | | | | |
| Male | 41 (60%) | 6 (75%) | 4 (67%) | 6 (43%) | — |
| Female | 27 (40%) | 2 (25%) | 2 (33%) | 8 (57%) | |
| Diabetes duration | 3.2 (1.5) | 2.1 (1.9) | 2.3 (1.7) | 1.6 (1.4) | 0.004 ~ |
| (years) | | | | | |
| HbA_{1C} (%) | 7.0 (0.5) | 7.6 (1.0) | 6.7 (0.6) | 6.9 (0.5) | 0.027 |
| BMI (kg/m²) | | | | | |
| Male | 28.5 (4.3) | 31.2 (5.8) | 30.3 (4.2) | 30.5 (2.5) | 0.403 |
| Female | 32.1 (5.5) | 25.8 (2.5) | 31.7 (5.2) | 31.1 (4.1) | 0.427 |
| Hip measurement | | | | | |
| (cm) | | | | | |
| Male | 106.6 (9.7) | 107.0 (11.3) | 106.0 (10.0) | 105.3 (3.8) | 0.989 |
| Female | 113.8 (12.3) | 97.0 (1.4) | 109.0 (9.9) | 114.1 (9.9) | 0.257 |
| Abdominal circ. | | | | | |
| (cm) | | | | | |
| Male | 104.2 (11.7) | 109.2 (13.8) | 106.5 (14.7) | 107.3 (3.7) | 0.735 |
| Female | 102.8 (13.2) | 84.5 (3.5) | 109.0 (4.2) | 106.3 (16.4) | 0.228 |
| Blood pressure | | | | | |
| Systolic (mmHG) | 144.2 (14.0) | 130.5 (15.0) | 125.3 (15.1) | 137.2 (17.5) | 0.003 *# |
| Diastolic | 84.3 (10.1) | 78.0 (8.4) | 73.7 (9.4) | 86.5 (10.5) | 0.024 #§ |
| (mmHG) | | | | | |
| Heart rate (1/min) | 69.6 (11.6) | 72.0 (10.7) | 68.3 (9.2) | 76.8 (6.4) | 0.147 ~§ |
| Albumin/creatinine | 3.3 (13.5) | 2.3 (2.6) | 2.6 (3.9) | 3.7 (4.8) | 0.994 |
| ratio (mg/mmol) | | | | | |
| Total chol. | 5.3 (1.1) | 5.1 (0.9) | 4.9 (1.1) | 4.7 (0.7) | 0.172 ~ |
| (mmol/l) | | | | | |
| Triglycerides | 2.1 (1.6) | 2.6 (1.1) | 1.6 (0.4) | 1.9 (0.9) | 0.681 |
| (mmol/l) | | | | | |
| HDL chol. | 1.3 (0.4) | 1.0 (0.3) | 1.1 (0.3) | 1.0 (0.3) | 0.010 ~ |
| (mmol/l) | | | | | |
| LDL chol. | 3.2 (0.8) | 3.0 (0.9) | 2.9 (1.0) | 2.8 (0.5) | 0.359 |
| (mmol/l) | | | | | |
| hsCRP (mg/l) | 2.8 (3.6) | 3.6 (4.7) | 1.0 (0.6) | 4.1 (5.9) | 0.527 |
| Plasma glucose | | | | | |
| (mmol/l) | 9.0 (1.7) | 10.6 (2.9) | 6.9 (1.2) | 8.0 (0.8) | 0.008 *#\hat{ } |
| Fasting | 10.9 (3.7) | 12.3 (5.5) | 7.7 (1.4) | 9.0 (1.6) | 0.162 |
| 2 h after test | | | | | |
| meal | | | | | |
| Leukocytes | 6.1 (1.6) | 6.2 (1.7) | 6.5 (2.1) | 7.4 (1.6) | 0.099 ~ |
| (GPtl/l) | | | | | |
| Insulin (mU/l) | | | | | |
| Fasting | 12.0 (8.0) | 10.7 (7.6) | 8.4 (5.0) | 13.8 (7.1) | 0.613 |
| 2 h after test | 58.8 (40.0) | 54.1 (40.5) | 49.9 (24.9) | 9.0 (1.6) | 0.162 |
| meal | | | | | |
| Proinsulin | | | | | |
| (pmol/l) | | | | | |
| Fasting | 10.0 (6.0) | 20.5 (28.3) | 6.5 (3.4) | 11.0 (9.5) | 0.051 |
| 2 h after test | 31.7 (17.9) | 51.2 (68.1) | 23.1 (7.1) | 32.6 (19.6) | 0.217 |
| meal | | | | | |
| HOMA | 47.6 (34.4) | 33.1 (20.5) | 52.1 (38.6) | 74.6 (33.0) | 0.215 $ |
| C-peptide (nmol/l) | 2.9 (1.1) | 3.2 (1.6) | 2.2 (0.8) | 2.0 (1.9) | 0.079 |

Sign. between Center 1 and 2
# Sign. between Center 1 and 3
~ Sign. between Center 1 and 3
\hat{ } Sign. between Center 2 and 3
$ Sign. between Center 2 and 4
§ Sign. between Center 3 and 4

### Analysis of Efficacy

TABLE 6
Change in the AUC 2hpp value after a test meal
(mean standard deviation)

| | | | Sign. visit 1 |
|---|---|---|---|
| | Visit 1 | Visit 8 | vs. visit 8 |
| Metformin group | 256.50 (72.837) | 213.61 (52.844) | 0.001 |
| Glargine group | 287.38 (67.235) | 224.38 (40.048) | 0.000 |

TABLE 7
Per protocol analysis of the primary end points
(mean standard deviation)

| | All patients | Metformin | Insulin | Sign. |
|---|---|---|---|---|
| | (n = 75) | (n = 36) | (n = 39) | M vs. I |
| AUC 2 h after test | 219.2 | 213.6 | 224.4 | 0.321 |
| meal - visit 8 | (46.6) | (52.8) | (40.0) | |
| AUC 2 h after | −53.4 (63.5) | −42.9 (73.1) | −63.0 (5.4) | 0.172 |
| test meal change | | | | |
| AUC day | 1990.0 | 1978.5 | 2000.3 | 0.774 |
| 2 - visit 8 | (322.9) | (337.8) | (313.1) | |
| AUC day | −550.6 | −416.1 | −671.2 | 0.039 |
| 2 - change | (534.2) | (537.6) | (507.9) | |
| AUC day | 2009.7 | 1972.5 | 2045.0 | 0.261 |
| 3 - visit 8 | (275.5) | (286.5) | (263.5) | |
| AUC day | −522.8 | −411.5 | −628.2 | 0.125 |
| 3 - change | (597.3) | (531.5) | (642.9) | |
| AUC 2 h | 59.3 (26.4) | 49.6 (25.0) | 68.3 (24.6) | 0.002 |
| incremental after | | | | |
| TM - visit 8 | | | | |
| AUC 2 h | −5.7 (36.3) | −5.8 (31.8) | −5.7 (40.4) | 0.989 |
| incremental after | | | | |
| TM - change | | | | |
| AUC between | −38.1 | −15.4 | −58.5 | 0.534 |
| 5.6 and fasting | (294.6) | (304.8) | (287.6) | |
| on day 2 - visit 8 | | | | |
| AUC between | −466.6 | −356.5 | −565.4 | 0.120 |
| 5.6 and fasting | (575.2) | (578.1) | (561.6) | |
| on day 2 - change | | | | |
| AUC between | 14.0 | 41.1 | −11.6 | 0.376 |
| 5.6 and fasting | (253.3) | (280.4) | (225.8) | |
| on day 3 - visit 8 | | | | |
| AUC between | −449.8 | −317.4 | −571.5 | 0.072 |
| 5.6 and fasting | (595.3) | (478.7) | (668.8) | |
| on day 3 - change | | | | |

TABLE 8
Laboratory and medical history data of the “per protocol” patients

| | All patients | Metformin | Insulin | Sign. |
|---|---|---|---|---|
| | (n = 75) | (n = 36) | (n = 39) | M vs. I |
| BMI - visit 8 | 29.6 (4.7) | 29.3 (5.3) | 29.9 (4.1) | 0.565 |
| BMI - change | 0.2 (1.5) | 1.0 (1.4) | −0.5 (1.3) | 0.000 |
| Abdomen - visit 8 | 102.5 (12.3) | 100.5 (13.7) | 104.3 (10.7) | 0.189 |
| Abdomen - change | 0.4 (4.2) | −2.0 (4.1) | 1.1 (3.7) | 0.001³ |
| Hip - 8th visit | 108.3 (10.4) | 108.0 (11.6) | 108.6 (9.4) | 0.813 |
| Hip - change | 0.1 (4.4) | −1.6 (4.1) | 1.6 (4.1) | 0.004³ |
| Total chol. - visit 8 | 5.2 (1.0) | 5.3 (1.0) | 5.2 (1.0) | 0.894 |
| Total chol. - change | −0.03 (0.7) | −0.02 (0.7) | −0.04 (0.8) | 0.946 |
| Glycemic | 8.7 (1.8) | 8.5 (2.1) | 8.8 (1.6) | 0.592 |
| load - visit 8 | | | | |
| Glycemic | −2.2 (2.3) | −1.7 (2.7) | −2.7 (1.9) | 0.065 |
| load - change | | | | |
| HbA1c - visit 8 | 6.3 (0.4) | 6.3 (0.3) | 6.4 (0.4) | 0.426 |
| HbA1c - change | −0.7 (0.6) | −0.6 (0.4) | −0.8 (0.7) | 0.297³ |
| HDL - visit 8 | 1.3 (0.4) | 1.3 (0.4) | 1.3 (0.3) | 0.272 |
| HDL - change | 0.1 (0.2) | 0.1 (0.2) | 0.1 (0.2) | 0.976 |
| HOMA - visit 8 | 93.6 (83.2) | 56.3 (34.5) | 127.9 (99.3) | <0.001² |
| HOMA - change | 42.4 (80.4) | 4.4 (19.5) | 77.2 (98.0) | <0.001³ |
| hsCRP - 8th visit | 3.1 (4.1) | 3.1 (5.2) | 3.1 (2.9) | 0.332² |
| hsCRP - change | −0.1 (2.7) | −0.2 (2.2) | 0.1 (3.0) | 0.062³ |
| LDL - visit 8 | 3.1 (0.8) | 3.1 (0.9) | 3.1 (0.8) | 0.871 |
| LDL - change | −0.1 (0.6) | −0.03 (0.6) | −0.1 (0.7) | 0.703 |
| MAGE - visit 8 | 7.2 (2.5) | 6.3 (2.4) | 8.0 (2.3) | 0.002 |
| MAGE - change | −0.6 (3.4) | −0.8 (3.3) | −0.4 (3.4) | 0.584 |
| Plasma glucose - | | | | |
| visit 8 | | | | |
| Fasting | 6.7 (1.2) | 7.2 (1.0) | 6.1 (1.1) | 0.000 |
| 2 h after test meal | 8.4 (2.3) | 8.4 (2.1) | 8.3 (2.5) | 0.832 |
| Plasma glucose - | | | | |
| change | | | | |
| Fasting | −2.3 (2.2) | −1.4 (1.5) | −3.1 (2.5) | 0.001 |
| 2 h after test meal | −2.2 (3.0) | −1.6 (2.5) | −2.8 (3.2) | 0.106 |
| Triglycerides | 2.1 (1.5) | 1.9 (0.8) | 2.2 (2.0) | 0.942² |
| (mmol/l) - visit 8 | | | | |
| Triglycerides | −0.2 (1.5) | −0.2 (1.1) | −0.2 (1.8) | 0.147³ |
| (mmol/l) - change | | | | |

²log-transformed for t-test
³tested in accordance with Mann-Whitney U test

TABLE 9
Parameters of beta-cell function

| | All patients | Metformin | Insulin | Sign. |
|---|---|---|---|---|
| | (n = 75) | (n = 36) | (n = 39) | M vs. I |
| Insulin - visit 8 | | | | |
| Fasting | 12.4 (7.9) | 10.0 (6.0) | 14.6 (8.8) | 0.011 |
| 2 h after test meal | 56.9 (46.5) | 50.8 (39.7) | 62.6 (51.8) | 0.416² |
| Insulin - change | | | | |
| Fasting | 0.1 (6.0) | −2.7 (3.6) | 2.7 (6.7) | <0.001 |
| 2 h after test meal | −1.8 (35.7) | −11.1 (30.2) | 6.5 (38.4) | 0.060³ |
| Proinsulin - visit 8 | | | | |
| Fasting | 5.6 (4.8) | 5.8 (4.6) | 5.4 (5.0) | 0.350² |
| 2 h after test meal | 23.7 (25.1) | 20.4 (19.9) | 26.8 (29.0) | 0.259² |
| Proinsulin - change | | | | |
| Fasting | −5.5 (8.6) | −3.2 (4.2) | −7.6 (10.8) | 0.001³ |
| 2 h after test meal | −9.1 (21.8) | −6.9 (14.5) | −11.1 (26.8) | 0.019³ |
| C-peptide - visit 8 | | | | |
| Fasting | 2.1 (1.1) | 2.7 (1.1) | 1.6 (0.9) | <0.001 |
| 2 h after test meal | 7.3 (3.3) | 7.9 (3.4) | 6.7 (3.1) | 0.115 |
| C-peptide - change | | | | |
| Fasting | −0.8 (1.1) | −0.3 (0.8) | −1.2 (1.1) | 0.001 |
| 2 h after test meal | −0.3 (2.2) | −0.1 (2.0) | −0.6 (2.4) | 0.348 |
| Insulin-proinsulin | | | | |
| ratio - visit 8 | | | | |
| Fasting | 20.7 (15.11) | 16.7 (17.4) | 24.5 (11.7) | <0.001 |
| 2 h after test meal | 22.6 (20.6) | 24.7 (27.1) | 20.7 (11.8) | 0.636² |
| Insulin-proinsulin | | | | |
| ratio - change | | | | |
| Fasting | 10.0 (12.4) | 2.9 (7.6) | 16.4 (12.4) | <0.001 |
| 2 h after test meal | 5.8 (9.3) | 3.0 (8.4) | 8.2 (9.4) | 0.015 |
| Insulin-C-peptide | | | | |
| ratio - visit 8 | | | | |
| Fasting | 54208.5 | 25456.9 | 80748.4 | <0.001² |
| | (52381.7) | (6469.3) | (61633.4) | <0.001 |
| 2 h after test meal | 51227.1 | 41915.2 | 59822.8 | |
| | (22497.9) | (14250.3) | (25311.2) | |
| Insulin-C-peptide | | | | |
| ratio - change | | | | |
| Fasting | −12657.6 | −25386.1 | 46799.3 | 0.002 |
| | (102124.9) | (116745.2) | (72879.7) | <0.001³ |
| 2 h after test meal | −31383.7 | −38956.8 | −24587.4 | |
| | (192054.3) | (184835.1) | (200473.7) | |
| Proinsulin-C-peptide | | | | |
| ratio - visit 8 | | | | |
| Fasting | 2868.0 | 2142.4 | 3537.8 | <0.001 |
| | (1745.7) | (1055.7) | (1987.2) | |
| 2 h after test meal | 3040.6 | 2458.1 | 3578.2 | 0.004² |
| | (2047.9) | (1417.4) | (2387.1) | |
| Proinsulin-C-peptide | | | | |
| ratio - change | | | | |
| Fasting | −1274.8 | −1386.3 | −1174.8 | 0.987³ |
| | (3191.0) | (3779.9) | (2599.1) | 0.211³ |
| 2 h after test meal | −2105.1 | −1668.7 | −2496.6 | |
| | (5535.0) | (4539.5) | (6331.2) | |

²log-transformed for t-test
³tested in accordance with Mann-Whitney U test

Before the start of the statistical analyses, all data were checked for consistency and the minimum, maximum and standard deviation and the normal distribution were checked. The normal distribution was checked with the Kolmogorov Smirnov test.

To analyze the difference between the two variables, the t-test was used. Variables with a non-normal distribution were logarithmically transformed before use. If a normal distribution could not be obtained, the Mann-Whitney U test was used.

Nominal variables were tested with Pearson's chi-squared test. The correlation was performed with the aid of Pearson's correlation coefficient.

The patients had the right to withdraw their consent to participation in the study at any time. When possible and the patient was willing, the concluding visit was carried out according to the same scheme as visit 8. In this case, the LOCF method (last observation carried forward) was applied.

Missing values and patients who dropped out were not replaced by “imputation methods”. Missing values were kept open and not used for statistical purposes

There were no interim analyses. A data monitoring committee was not required.

On the whole, there were no drastic differences between the study sites. The few significant differences can be explained by the low number of patients at study sites 2, 3 and 4.

### EXAMPLE 7

### Summary of the Results

GLA vs. MET treatment revealed a distinct reduction in the FPG level (Δ: 3.1±2.5 vs. 1.4±1.5 mmol/L; p<0.001), a decrease in the interstitial glucose level during CGM (Δ: 2.4±1.8 vs. 1.4±1.8 mmol/L; p=0.02) and the HbA1c level (7.2 to 6.4% vs. 6.9 to 6.3%; n.s). The total area under the curve (AUC-IG) fell by 671.2±507.9 in the case of GLA vs. 416.1±537.6 mmol/L min in the case of MET (p=0.04). The levels of the postprandial plasma glucose two hours after a test meal and the differences in the area under the curve (AUC-IG) of continuous glucose monitoring (GCM) after said test meal showed no significance. A significant reduction in the ratio of proinsulin to C-peptide was found, indicating an improvement in beta-cell function in each case after treatment with GLA and MET. The mean dose of GLA at the last observation time (“LOCF”) was 0.3±0.1 U/kg. Patients who received GLA gained 1.5±3.8 kg in weight compared to a weight loss of 3.0±4.3 kg in the case of use of MET (p<0.001). For a HbA1c level of 6.3%, there were equivalent hypoglycemic events in both treatment groups; there were also equivalent periods at which the measured level with regard to interstitial glucose was less than 3.9 mmol/L during continuous glucose monitoring (GCM).

### EXAMPLE 8

### Conclusions

Early treatment of type 2 diabetes patients with GLA led to more effective control of the so-called “fasting glucose” level and of the overall glycemic situation with an improvement in beta-cell function compared to the standard treatment with MET. There was no rise in the number of hypoglycemic events.

### LITERATURE

1. Nathan D M, Buse J B, Davidson M B, Ferrannini E, Holman R R, Sherwin R, Zinman B. Management of hyperglycaemia in type 2 diabetes mellitus: a consensus algorithm for the initiation and adjustment of therapy. Update regarding the thiazolidinediones. Diabetologia. 2008; 51(1):8-11.
2. [No authors listed] Effect of intensive blood-glucose control with metformin on complications in overweight patients with type 2 diabetes (UKPDS 34). UK Prospective Diabetes Study (UKPDS) Group. Lancet. 1998; 352(9131):854-65.
3. Widjaja A, Stratton 1M, Horn R, Holman R R, Turner R, Brabant G. UKPDS 20: plasma leptin, obesity, and plasma insulin in type 2 diabetic subjects. J Clin Endocrinol Metab. 1997; 82(2):654-7.
4. Hanefeld M, Koehler C, Hoffmann C, Willhelm K, Kamke W, Gerstein H. Importance of fasting glucose control for glycemic variability—a substudy of the ORIGIN (Outcome Reduction with Initial Glargine Intervention) trial with continuous glucose measurement. In preparation
5. Monnier L, Colette C, Dunseath G J, Owens D R. The loss of postprandial glycemic control precedes stepwise deterioration of fasting with worsening diabetes. Diabetes Care. 2007; 30(2):263-9.
6. Ott P, Benke I, Stelzer J, Köhler C, Hanefeld M. Multifaktorielle Intervention bei Typ 2-Diabetes (Multifactorial intervention in type 2 diabetes): “Diabetes in Germany” (DIG) Studie—eine prospektive 4-Jahres-Studie zur Therapiequalitat (“Diabetes in Germany” (DIG) study—a prospective 4-year study on therapy quality). DMW 2008, submitted.
7. Rao A D, Kuhadiya N, Reynolds K, Fonseca V A. Is the combination of sulfonylureas and metformin associated with an increased risk of cardiovascular disease or all-cause mortality?: a meta-analysis of observational studies. Diabetes Care. 2008; 31(8):1672-8.
8.-
9. Guideline for management of postmeal glucose, International Diabetes Federation 2007, Belgium, www.idf.org
10. Hanefeld M. Normnahe postprandiale Hyperglykämie-eine essenzielle Komponente guter Diabeteskontrolle und Prävention kardiovaskulärer Erkrankungen (Near-normal postprandial hyperglycemia—an essential component of good diabetes control and prevention of cardiovascular diseases). Paul Langerhans lecture 2007. Diabetologie und Stoffwechsel 2007; 2:362-369.
11. Hanefeld M, Temelkova-Kurktschiev T. The postprandial state and the risk of atherosclerosis. Diabet Med. 1997; 14 Suppl 3:S6-11.
12. Holman R R, Paul S K, Bethel M A, Matthews D R, Neil H A. 10-Year Follow-up of Intensive Glucose Control in Type 2 Diabetes. N Engl J. Med. 2008; 359(15):1577-1589.
13. Action to Control Cardiovascular Risk in Diabetes Study Group, Gerstein H C, Miller M E, Byington R P, Goff D C Jr, Bigger J T, Buse J B, Cushman W C, Genuth S, Ismail-Beigi F, Grimm R H Jr, Probstfield J L, Simons-Morton D G, Friedewald W T. Effects of intensive glucose lowering in type 2 diabetes. N Engl J. Med. 2008; 358(24):2545-59.
14. Patel A; ADVANCE Collaborative Group, MacMahon S, Chalmers J, Neal B, Woodward M, Billot L, Harrap S, Poulter N, Marre M, Cooper M, Glasziou P, Grobbee D E, Hamet P, Heller S, Liu L S, Mancia G, Mogensen C E, Pan C Y, Rodgers A, Williams B. Effects of a fixed combination of perindopril and indapamide on macrovascular and microvascular outcomes in patients with type 2 diabetes mellitus (the ADVANCE trial): a randomised controlled trial. Lancet. 2007; 370(9590):829-40.
15. VADT Presentation ADA 2008
16. Kahn S E, Haffner S M, Heise M A, Herman W H, Holman R R, Jones N P, Kravitz B G, Lachin J M, O'Neill M C, Zinman B, Viberti G; ADOPT Study Group. Glycemic durability of rosiglitazone, metformin, or glyburide monotherapy. N Engl J. Med. 2006; 355(23):2427-43.
17. Knowler W C, Barrett-Connor E, Fowler S E, Hamman R F, Lachin J M, Walker E A, Nathan D M; Diabetes Prevention Program Research Group. Reduction in the incidence of type 2 diabetes with lifestyle intervention or metformin. N Engl J. Med. 2002; 346(6):393-403.

### LIST OF ABBREVIATIONS

2hppG glucose level 2 hours after the start of the test meal
a acute
ADA American Diabetes Association
ADMA Asymmetric Dimethylarginine
AE Adverse Event
ALAT Alanine Aminotransferase
AMG German Medicinal Products Act
ASAT Aspartate Aminotransferase
AR Adverse Reaction
AUC TM Area Under the Curve during Test Meal
BfArM German Federal Institute for Drugs and Medical Devices
BG Blood Glucose
CGM Continous Glucose Monitoring
CRA Clinical Research Associate
CRF Case Report Form
CTCAE Common Terminology Criteria for Adverse Events
DDG German Diabetes Society
DSMB Data Safety Monitoring Board
EASD European Association for the Study of Diabetes
FAS Full Analysis Set
FFA Free Fatty Acids
FG Fasting Glucose before test meal
FPFV First Patient First Visit
GCP Good Clinical Practice
gGT gamma-Glutamyl Transpeptidase
h hour
i. v. intravenous
ICH International Conference on Harmonization
IDF International Diabetes Federation
ISF Investigator Site File
LKP Head of the Clinical Trial
LOCF Last Observation Carried Forward
LPLV Last Patient Last Visit
MAGE Mean Amplitude of Glycemic Excursions
MMP Matrix Metalloproteinases
n number of patients
NA Not Applicable
FBG Fasting Blood Glucose
ND Not Done
OAD Oral Antidiabetic
PAI-1 Plasminogen Activator Inhibitor 1
Peak maximum glucose level during the test meal
p. o. per os
pp postprandial
PPS Per Protocol Analysis Set
SAE Serious Adverse Event
SAP Statistical Analysis Plan
SAR Serious Adverse Reaction
SAS Statistical Analysis System
SD Standard Deviation of the mean
SDV Source Data Verification
SMBG Self Monitoring of Blood Glucose
SOP Standard Operating Procedure
SPSS Statistical Package for the Social Sciences
SUSAR Suspected Unexpected Serious Adverse Reaction
TM Test Meal
TMF Trial Master File
UAR Unexpected Adverse Reaction

1. A method for treating type 2 diabetes patients, wherein insulin glargine is administered to the patient in an amount clinically tested to be safe and effective, wherein the type 2 diabetes is early type 2 diabetes.

2. The method as claimed in claim 1, wherein the insulin glargine is used as first-line drug.

3. The method as claimed in either of the preceding claims, wherein the diagnosis of the patient with type 2 diabetes is less than five years before the start of the treatment with insulin glargine.

4. The method as claimed in any of the preceding claims, wherein the HbA1c level of the patient at the time of the diagnosis is between approximately 6.5 to approximately 8.5% inclusive.

5. The method as claimed in any of the preceding claims, wherein the dose of insulin glargine is determined according to a standardized titration algorithm for a target value of the fasting glucose level (FPG) of approximately 5.6 mmol/l or less.

6. A method for treating type 2 diabetes patients, wherein insulin glargine is administered as first-line drug to the patient in an amount clinically tested to be safe and effective, wherein the patient would have received an oral antidiabetic as first-line drug in a standard treatment.

7. The method as claimed in claim 6, wherein the diagnosis of the patient with type 2 diabetes is less than five years before the start of the treatment with insulin glargine.

8. The method as claimed in either of claims 6 and 7, wherein the HbA1c level of the patient at the time of the diagnosis is between approximately 6.5 to approximately 8.5% inclusive.

9. The method as claimed in any of claims 6, 7 and 8, wherein the dose of insulin glargine is determined according to a standardized titration algorithm for a target value of the fasting glucose level (FPG) of approximately 5.6 mmol/l or less.

10. The method as claimed in any of claims 6, 7, 8 and 9, wherein the oral antidiabetic is selected from a group comprising metformin.

11. A method for treating type 2 diabetes patients, wherein insulin glargine is administered to the patient in an amount clinically tested to be safe and effective, wherein the patient had not yet received beforehand any insulin or insulin analog or oral antidiabetic.

12. The method as claimed in claim 11, wherein the diagnosis of the patient with type 2 diabetes is less than five years before the start of the treatment with insulin glargine.

13. The method as claimed in either of claims 11 and 12, wherein the HbA1c level of the patient at the time of the diagnosis is between approximately 6.5 to approximately 8.5% inclusive.

14. The method as claimed in any of claims 11, 12 and 13, wherein the dose of insulin glargine is determined according to a standardized titration algorithm for a target value of the fasting glucose level (FPG) of approximately 5.6 mmol/l or less.

15. The method as claimed in any of claims 11, 12, 13 and 14, wherein the oral antidiabetic is selected from a group comprising metformin.

16. The method as claimed in any of claims 11, 12, 13, 14 and 15, wherein the insulin or insulin analog is selected from a group comprising human insulin, insulin glargine, insulin detemir and insulin degludec.

17. A product comprising a packing material, insulin glargine and a label and/or a package leaflet in which it is indicated that patients who are being treated with insulin glargine can receive said treatment for early type 2 diabetes.

18. The product as claimed in claim 17, wherein the label and/or the package leaflet indicate that insulin glargine can be used as first-line drug.
